(19) United States
(12) Patent Application Publication
(10) Pub. No.: US 2006/0014232 A1
(43) Pub. Date: Jan. 19, 2006

(54) Immobilization method

(75) Inventors: Junichi Inagawa, Yokohama-shi (JP); Noriyuki Inomata, Ninomiya-machi (JP)

Correspondence Address: SEED INTELLECTUAL PROPERTY LAW GROUP PLLC, 701 FIFTH AVE, SUITE 6300, SEATTLE, WA, 98104-7092, US

(73) Assignees: Biacore AB, Uppsala (SE); Reverse Proteomics Research Institute Co., Ltd., Kisarazu (JP)

(21) Appl. No.: 10/535,736

(22) PCT Filed: Sep. 22, 2003
(86) PCT No.: PCT/SE03/01473

§ 371 Date: May 18, 2005

(30) Foreign Application Priority Data
Nov. 19, 2002 (JP) 2002-335334

(51) Int. Cl. C12Q 1/37; C12N 11/04

(52) U.S. Cl. 435/23; 435/182

(57) ABSTRACT

Various biomolecules can be firmly immobilized to a substrate by a method which comprises contacting a solution containing biomolecules provided with at least one tag with an immobilization substrate which has (i) binding sites for the biomolecule tag or tags, and (ii) activated reactive groups which are capable of forming covalent bonds with the biomolecules, to covalently bind the biomolecules interacting with the tag binding sites to the immobilization substrate.

### FIELD OF THE INVENTION

The present invention relates to a method for immobilizing biomolecules, which can be widely used, for example, when immobilizing proteins on the surface of a substrate.

### BACKGROUND OF THE INVENTION

Information concerning interactions between proteins and drugs, or protein-protein interactions is analyzed by various methods, and is extremely important for the development of novel drugs and the improvement of the action of existing drugs, and for the reduction of side-effects. In recent years, equipment, which applies the principle of surface plasmon resonance (SPR) to analyze interactions in real time without the use of radioisotopes, such as Biacore® 3000 (Biacore AB, Uppsala, Sweden) or the like is being used.

In the analysis of interactions using the SPR principle, one component of the protein-protein interaction to be analyzed, or one component of the protein-drug interaction to be analyzed is immobilized onto a sensor chip. Then, the other component, the protein or the drug, is made to react on the sensor chip, the mass change brought about by the protein-protein or protein-drug interaction then being detected as an SPR signal.

In the case of immobilizing a low molecular weight compound, such as a drug or the like, it is necessary to modify an appropriate site on the low molecular weight molecule for immobilization, and it is necessary to select the site of modification carefully so that the modification does not have an adverse influence on the binding to the proteins. Further, various lengths of modified molecules are prepared and tested so that the molecular structure of the modified site has an appropriate length for the interaction analysis.

Alternatively, in the case of immobilizing a protein, the two main ways of carrying this out are:
(A) a method where the immobilization is carried out by coupling the protein rigidly to the sensor chip with a covalent bond, and
(B) a method where the affinity between the sensor chip and the protein is utilized to bind the protein mildly to the sensor chip.

As method (A) are known: 1) a method whereby an amino group of the protein and a carboxyl group of the sensor chip are coupled (the amine coupling method); 2) a method whereby a carboxyl group of the protein is modified by 2-(2-pyridinyldithio)-ethaneamine (PDEA), while a carboxyl group of the sensor chip is modified to a thiol group (—SH) so that the two are coupled via an S—S bond (the surface thiol coupling method); and 3) a method whereby the sensor chip is modified with PDEA or the lice and coupled by forming an —S—S— bond with a free —SH group on the protein (the ligand thiol coupling method).

As method (B) are known: 1) a method whereby a histidine tag (His-tag) is introduced into the protein and then bound to a sensor chip coated with nitrilotriacetic acid (NTA) through Ni²⁺; and 2) a method whereby various antibodies are immobilized on the sensor chip, and the corresponding antigens are then bound to the antibodies and thereby immobilized on the sensor chip.

In method (A), any amino group or carboxyl group of the protein may be modified by the immobilization, but in many cases good affinity is still preserved. In method (B), on the other hand, it is necessary to add a sequence capable of expressing an affinity site, such as a His-tag, an antigen peptide or the like, to a part of the gene for the protein by a recombinant DNA technique, but when carrying out the immobilization it is then not necessary to modify the protein.

In this way, it is in general possible to carry out immobilization of a protein more easily than immobilization of low molecular weight compounds. For this reason, much research is carried out by immobilizing a protein to a sensor chip to analyze the interactions.

However, in method (A), when immobilizing the protein, whether by the amine coupling method, the surface thiol coupling method, or any other method, it is necessary to concentrate the protein on the sensor chip. Without this concentration (preconcentration), it is in general almost impossible to immobilize a protein. Preconcentration can be carried out by dissolving or diluting the protein at the time of coupling in a buffer solution whose pH is slightly lower than the isoelectric point (pI) and whose ionic strength is weak (approximately 10 mM sodium acetate buffer solution or the like). In other words, in a buffer solution whose pH is below the pI of the protein, the protein has a disposition for the total electrical charge to be positive, and at the same time since the carboxyl group on the sensor chip is negatively charged from an alkaline state to an approximately pH 3.5 acidic state, the protein will be concentrated on the sensor chip by an electrostatic force. Through this preconcentration effect, in spite of using physiological concentrations of protein, it is possible to achieve high concentrations of protein on the sensor chip, with the result that high amounts of immobilization can be achieved.

Furthermore, since the protein is firmly immobilized on the sensor chip by a covalent bond, once the protein has been immobilized, it maintains a stable bond to the sensor chip and can be used repeatedly for the analysis of interactions.

However, in order to achieve this preconcentration effect, it is necessary to expose the protein to low pH conditions and to a buffer solution where the ionic strength is also lower than normal physiological conditions. Further, since many acidic proteins do not have a positive total electrical charge even at around pH 4.0, the preconcentration effect is not achieved, and as a result it is not possible to immobilize the protein.

In the surface thiol coupling method, as a result of PDEA modification of the carboxyl group on the protein, this method achieves the preconcentration effect by reducing the negative (minus) charges on the protein, thus causing the pI to rise. Therefore a good result has been achieved using the surface thiol coupling method even with a number of acidic proteins, but with this method it is necessary to modify the protein with PDEA or the like, after which it is necessary to carry out a purification. Because of this, the amount of protein required is approximately 100 μg, or about a hundredfold more than the approximately 1 μg required for the amine coupling method. Further, since coupling is carried out through the S—S bond in the surface thiol coupling method, and since alkaline solutions cannot be used for washing of the immobilized protein and regeneration operations at the time of the interaction analysis, proteins which require an alkaline solution for regeneration can be immobilized on the sensor chip, but in fact interaction analysis cannot be carried out.

In contrast, with method (B), the buffer solution used when immobilizing a His-tagged protein where the His-tag has been inserted into the protein by a recombinant DNA technique can be a buffer solution of physiological conditions (PBS or the like). However, the affinity of the bond between NTA and the protein is generally weak, and although the protein has been immobilized on the sensor chip through the Ni²⁺ ion, the protein may gradually separate from the sensor chip after the immobilization. Further, the binding between a His-tagged protein and the NTA sensor chip becomes increasingly unstable at high salt concentration, at low salt concentration, at acidic pH conditions, and at alkaline pH conditions, making it impossible to carry out interaction analysis where sensor chip washing and regeneration operations are required.

As mentioned above, in the case of using the amine coupling method, there exists the problem that there is a limitation as to the proteins that can be immobilized, such as that acidic proteins cannot be immobilized. Further, in the case of using the surface thiol coupling method, although it is possible that many acidic proteins can be immobilized, the problems that large amounts of protein are necessary, and that alkaline washing and regeneration operations are not possible still remain. Further, in the case of using a His-tag, a wide variety of His-tagged proteins can be immobilized on the sensor chip, but there exist the problems that the bindings are unstable and gradually dissociate, and also that at the time of interaction analysis a protein that requires washing and regeneration operations of the immobilized protein cannot be analyzed.

The object of the present invention, in view of the circumstances mentioned above, is to provide a protein immobilization method which can immobilize various biomolecules, such as proteins, and which will immobilize them firmly to the substrate.

### SUMMARY OF TE INVENTION

In order to achieve the above-mentioned object, as a result of serious deliberations on the part of the inventors, this invention was completed by discovering that various proteins (and other biomolecules) can be strongly immobilized if, when the protein is to be immobilized on a substrate, after activating a reactive group on the immobilization substrate, the reactive group is reacted with a protein that has a tag, causing the protein tag and the immobilization substrate to interact, whereby it is possible to cause a covalent bond to form between the protein and the immobilization substrate.

In a first aspect, the present invention provides a method for immobilizing biomolecules, such as proteins, which method comprises contacting a solution containing a biomolecule or biomolecules provided with at least one tag with an immobilization substrate which has (i) binding sites for the biomolecule tag or tags, and (ii) activated reactive groups which are capable of forming covalent bonds with the biomolecule or biomolecules.

In one embodiment, the method comprises:
a first step of activating reactive groups of an immobilization substrate which has reactive groups capable of forming a covalent bond with the biomolecule or biomolecules, such as proteins, to be immobilized which have a tag(s);
a second step of reacting a solution containing the biomolecule or biomolecules to be immobilized with the immobilization substrate following the first step, and
wherein, in the second step, the biomolecule or biomolecules are immobilized on the immobilization substrate through an interaction taking place between the tag(s) and a tag-binding site of the immobilization substrate and a covalent bond formed between the reactive groups and the biomolecule or biomolecules.

The reactive groups may, for example, be carboxyl groups, and, in the second step, an amine coupling is effected between the carboxyl groups and amine groups on the biomolecules, such as proteins, to be immobilized.

The tag may, for example, be a histidine tag, and in the second step, an interaction takes place between the histidine tag and the immobilization substrate.

The interaction in the second step may then take place between the histidine tag and the immobilization substrate through a complex, preferably a metal ion chelate, for example, Ni²⁺-nitrilotriacetic acid (Ni-NTA) or Ni²⁺-iminodiacetic acid (Ni-IDA).

Alternatively, the tag-binding site of the immobilization substrate in the second step may be an antibody to the tag.

In such a case, the tag is preferably a histidine tag, the antibody is anti-histidine antibody and, in the second step, an interaction takes place between the histidine tag and the immobilization substrate through the anti-histidine antibody.

The tag may also be an inherent part of a native biomolecule.

In a second aspect, the present invention provides a method for determining biomolecule-low molecular weight compound affinity and/or kinetics comprising:
a step for reacting a sample containing a low molecular weight compound(s) to be determined with an immobilization substrate to which a biomolecule(s) have been immobilized using the method for immobilizing biomolecules, such as proteins, according to the first method aspect above, and
a step for determining the affinity and/or kinetics of the low molecular weight compound(s) contained in the sample and the biomolecule(s) immobilized on the immobilization substrate.

The determination of affinity may comprise determining association and/or dissociation constants, and the determination of kinetics may comprise determining association rate constants and/or dissociation rate constants.

In one embodiment of the method for determining biomolecule-low molecular weight compound affinity and/or kinetics, the affinity and/or kinetics of the biomolecules, such as proteins, and the low molecular weight compounds is determined using the principle of surface plasmon resonance (SPR) in the step for determining affinity and/or kinetics.

In a third aspect, the invention provides a method for determining protein-protein affinity and/or kinetics comprising:
a step for reacting a sample containing a protein(s) to be determined with an immobilization substrate to which a protein(s) have been immobilized using the method for immobilizing biomolecules according to the first method aspect above, and
a step for determining the affinity and/or kinetics of the protein(s) contained in the sample and the protein(s) immobilized on the immobilization substrate.

In one embodiment of the method for determining protein-protein affinity and/or kinetics, the affinity of the protein(s) in the sample and the immobilized protein(s) is determined using the principle of surface plasmon resonance (SPR) in the step for determining affinity and/or kinetics.

In a fourth aspect, the present invention provides an immobilization substrate comprising immobilized biomolecules, such as proteins, which are immobilized according to the method for immobilizing biomolecules according to the first method aspect above.

In a preferred embodiment, the immobilization substrate comprises:
a substrate, and
polysaccharide chains arranged on the substrate, into which are introduced reactive groups capable of forming a covalent bond with a biomolecule(s) to be immobilized thereon, and
the immobilization substrate is characterized in that the biomolecule(s) interact with the polysaccharide chains through a chelate and form covalent bonds with the reactive groups.

The above and other aspects of the invention will be evident upon reference to the accompanying drawings and the following detailed description.

### BRIEF DESCRIPTION OF THE DRAWINGS

FIG. 1 is a cross section of the relevant parts of a sensor chip produced to be appropriate for the method for the immobilization of biomolecules of the present invention.

FIG. 2 is a conceptual configuration diagram for explaining the configuration of analytical equipment using the SPR principle.

FIG. 3 is a characteristic diagram showing the relationship between time and response for various pH values of a protein solution.

FIG. 4 is a characteristic diagram showing a sensorgram for an HSA immobilization operation.

FIG. 5 is a characteristic diagram showing a sensorgram for an N-terminal His-tagged COX-2 immobilization operation as carried out in comparative example 3.

FIG. 6 is a characteristic diagram showing a sensorgram for an N-terminal His-tagged FKBP immobilization operation as carried out in comparative example 4.

FIG. 7 is a characteristic diagram showing the results of a measurement of an interaction between N-terminal His-tagged COX-2 and NS-398 as carried out in comparative example 5.

FIG. 8 is a characteristic diagram showing the results of a measurement of the binding between N-terminal His-tagged FKBP and FK506 as carried out in comparative example 6.

FIG. 9 is a characteristic diagram showing a sensorgram for an operation to immobilize N-terminal His-tagged FKBP on an NTA sensor chip as carried out in practical example 1.

FIG. 10 is a characteristic diagram showing a sensorgram for an operation to immobilize N-terminal His-tagged COX-2 on an NTA sensor chip as carried out in practical example 2.

FIG. 11 is a characteristic diagram showing a sensorgram for an operation to immobilize N-terminal His-tagged Cyclophilin A on a CM5 sensor chip as carried out in practical example 3.

FIG. 12 is a characteristic diagram showing a sensorgram for an operation to immobilize N-terminal His-tagged Akt1/PKBa on an NTA sensor chip as carried out in practical example 4.

FIG. 13 is a characteristic diagram showing a sensorgram for an operation to immobilize N-terminal His-tagged MSK1 on an NTA sensor chip as carried out in practical example 5.

FIG. 14 is a characteristic diagram showing a sensorgram for an operation to immobilize N-terminal His-tagged PKA on an NTA sensor chip as carried out in practical example 6.

FIG. 15 is a characteristic diagram showing a sensorgram for an operation to immobilize N-terminal His-tagged PRAK on an NTA sensor chip as carried out in practical example 7.

FIG. 16 is a characteristic diagram showing a sensorgram for an operation to immobilize N-terminal His-tagged ROKα/ROCK-II on an NTA sensor chip as carried out in practical example 8.

FIG. 17 is a characteristic diagram showing the result of a measurement of the binding between N-terminal His-tagged FKBP and FK506 as carried out in practical example 9.

FIG. 18 is a characteristic diagram showing the result of a measurement of the binding between N-terminal His-tagged COX-2 and NS-398 as carried out in practical example 10.

FIG. 19 is a characteristic diagram showing the result of a measurement of the binding between N-terminal His-tagged Cyclophilin A and Cyclosporine A as carried out in practical example 11.

FIG. 20 is a characteristic diagram showing a sensorgram for an operation to immobilize mouse IgG on a sensor chip with protein A as capture molecule as carried out in practical example 12.

FIG. 21 is a characteristic diagram showing a sensorgram for an operation to immobilize mouse IgG on a sensor chip without capture molecule as carried out in practical example 12.

FIG. 22 is a characteristic diagram showing two superposed sensorgrams for (i) an operation to bind anti-mouse IgG to a sensor chip having mouse IgG immobilized via protein A, and (ii) an operation to bind anti-mouse IgG to a sensor chip having mouse IgG immobilized without protein A as carried out in practical example 13.

### DETAILED DESCRIPTION OF THE INVENTION

The method for the immobilization of biomolecules, such as proteins, of the present invention can be applied when immobilizing a biomolecule to an immobilization substrate, and is not limited to applications for any specific technical area.

For example, the method for the immobilization of biomolecules of the present invention may be applied for preparing a sensor chip with an immobilized biomolecule(s) for use with analysis by the use of a variety of detection methods including both label-free methods and methods requiring a label, such as a fluorophore or a chromophore. Label-free methods include those based on evanescent wave sensing, such as, for example, the surface plasmon resonance (SPR) principle. Of other label-free detection techniques, for example, the principle of Quartz-Crystal Microbalance (QCM) may be mentioned.

Further, the method for the immobilization of biomolecules of the present invention may, for example, also be applied when preparing so-called protein chips (protein arrays) or affinity beads (affinity columns).

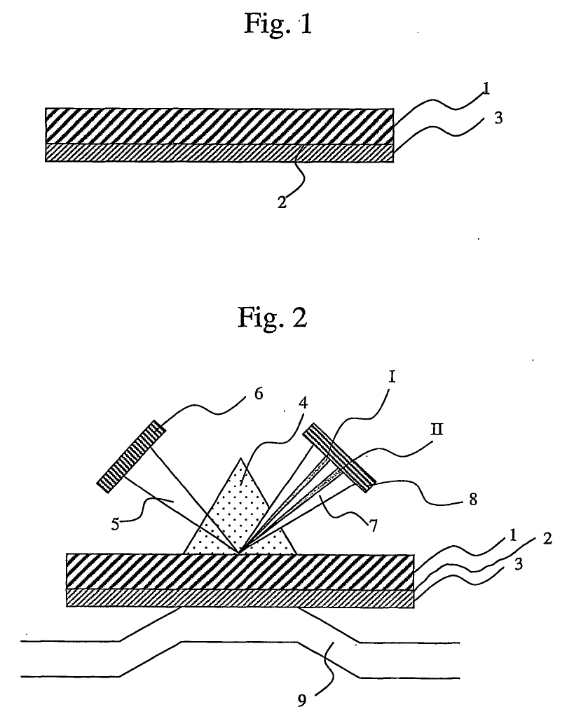

Hereinafter, the technique will be described through the example of sensor chips for use with analysis using the SPR principle. As shown in FIG. 1, the sensor chip comprises a transparent base material 1, a metal coating 2 affixed to one principal surface thereof, and an immobilization substrate or matrix 3 affixed to the metal coating 2. The immobilization matrix 3 may e.g. be a self-assembled monomolecular monolayer (SAM) which has a reactive group such as a carboxyl group, or a SAM and carboxymethyldextran immobilized on the metal coating 2.

The immobilization matrix 3 comprises a reactive group(s) which forms a covalent bond with a protein to be immobilized. The reactive group on the immobilization matrix 3 means a functional group which is capable of forming a covalent bond with the biomolecule to be immobilized. As regards the reactive group, for example, a carboxyl group or a thiol group may be mentioned. Further, the immobilization matrix 3 comprises a tag binding site(s) to which a tag of the biomolecule to be immobilized can bind. The tag binding site may be selected to be appropriate for the above-mentioned tag, but for example, for a protein with a histidine tag, nitrilotriacetic acid (NTA) may be mentioned, for a protein with a glutathion S-transferase tag, glutathion, and for a protein with a maltose binding protein tag, maltose. Further, for proteins with an antigen peptide tag, an antibody which has an antigen-antibody reaction with the antigen peptide can be used as the tag binding site.

Further, in the method for the immobilization of biomolecules of the present invention, any biomolecule may be used as the bimolecule to be immobilized in the method with no limitation as long as it comprises a tag as defined above. As referred to here, the tag is a site which interacts with the tag binding site on the immobilization substrate 3 and contributes to a bond between the biomolecule, e.g. a protein, and the immobilization substrate 3. For example, the following may be mentioned as tags: histidine tag (hereinafter His-tag; a His tag comprises at least two, e.g. 5-6 histidine residues which usually are consecutive but also may be interrupted by another amino acid(s)), glutathion S-transferase tag (hereinafter GST-tag), maltose binding protein tag (hereinafter MBP-tag), and antigen peptide tag, or the like. The antigen peptide tag is a peptide on which there is an antigen, and which is used as a tag. The following specific examples may be mentioned: His-tag, His G-tag, HA-tag, FLAG-tag, NS1(81)-tag, green fluorescent protein (GFP)-tag, IRS-tag, LexA-tag, Thioredoxin-tag, Polyoma virus medium T antigen epitope-tag, SV40 Large T Antigen-tag, Paramoxyvirus SV5-tag, Xpress-tag, GST-tag, MBP-tag, or the like. The tag may also be an inherent part of a native biomolecule, such as e.g. the Fc-part of IgG which can bind to immobilized protein A or G.

As mentioned above, there is no limitation with regard to the biomolecule, and any biomolecule with any attributes or properties can apply to the method, including both native and synthetically produced molecules, provided that the biomolecule has a functional group(s) that can bind to the reactive groups of the immobilization substrate. The biomolecule is preferably a protein or a polypetide, but may also be e.g. a carbohydrate, lipid or nucleic acid. Especially with regard to proteins, they may be basic proteins or acidic proteins, or they may be hydrophobic proteins or hydrophilic proteins.

A protein that has a tag, for example, can be prepared by transforming a host using an expression vector which has a gene that codes for the tag and a gene that codes for the protein in a state where the frames match, causing the protein to be expressed as a fusion protein of the tag and the protein within the genetic transformation cell, and then recovering the fusion protein.

The method for the immobilization of biomolecules, such as proteins, according to the present invention may be carried out in the following way. Firstly, the reactive group on the immobilization substrate is activated. Activation means transforming the reactive group into a state where it is capable of forming a covalent bond with a protein to be immobilized which exists in proximity of the reactive group. For example, by reacting an immobilization substrate 3, which has a carboxyl group as the reactive group, with a mixed solution of N-ethyl-N′-(dimethylaminopropyl)carbodiimide (EDC) and N-hydroxysuccinimide (NHS), the carboxyl group can be activated.

Next, the protein to be immobilized is reacted with the immobilization substrate 3, causing the tag on the protein to be immobilized to interact with the immobilization substrate 3. As referred to here, interaction means the binding between the tag and the tag binding site, the protein and the immobilization substrate 3 thereby forming a comparatively weak bond. For example, in the case that a protein has a His-tag as the tag, a metal such as nickel is trapped in NTA which has been introduced onto the immobilization substrate 3, and the His-tag and the NTA form a complex through the nickel. The nickel may be trapped in the NTA either before or after the activation of the immobilization substrate 3. In this way, the protein with the His-tag and the immobilization substrate 3 which has had NTA introduced thereon can be made to interact.

Further, in the case that a protein has a GST-tag as the tag, an immobilization substrate onto which glutathion has been introduced and the protein can be made to interact by having them coexist in a physiological phosphate buffer (for example PBS) or a physiological buffer solution based on Hepes (for example HBS). Further, in the case where a protein with an antigen peptide and an immobilization substrate 3 onto which an antibody has been introduced are used, these may also be made to interact in the same way by having them coexist in a physiological phosphate buffer (for example PBS) or a physiological buffer solution based on Hepes (for example HBS).

In the method for the immobilization of biomolecules, such as proteins, of the present invention, as mentioned above, in order to cause the tag on the biomolecule to be immobilized to interact with the immobilization substrate 3, the biomolecule to be immobilized should be present in the proximity of the immobilization substrate 3 in a comparatively high concentration. This induces a state where covalent bonds are easily formed between the activated reactive group and the biomolecule, and covalent bonds are thus easily formed between the activated reactive group and the biomolecule.

For example, in the case where the reactive group is a carboxyl group, a covalent bond is formed between an amino group on a protein to be immobilized and the reactive group, i.e. amine coupling is effected. Further, in the case that a carboxyl group is the reactive group, by modifying the carboxyl group with PDEA (2-(2-pyridinyldithio) ethaneamine hydrochloride), a covalent bond is formed between a free thiol group on a protein to be immobilized and the reactive group, i.e. ligand thiol coupling is effected. Further, in the case that the protein to be immobilized has a carboxyl group, it can first be reacted with PDEA to modify the carboxyl group with PDEA. After activating the carboxyl group on the immobilizing substrate 3, this carboxyl group can then be transformed into a thiol group by reacting it with cystamine dihydrochloride, and then reducing it with dithiothreitol (DTT). Thereby a covalent bond (disulfide bond) is formed between the PDEA modified carboxyl group of the protein and the thiol group on the immobilized substrate 3. In other words, surface thiol coupling is effected.

In this way, by interaction between the tag and the tag binding site, and the formation of a covalent bond between the reactive group and the biomolecule, the biomolecule can be immobilized on the immobilization substrate. Thus, by use of the method of the present invention, it is possible to arrange for the biomolecule to be proximate to the immobilization substrate 3 in a comparatively high concentration by causing the tag and the tag binding site to interact. Because of this, the method for immobilizing biomolecules according to the present invention permits the formation of covalent bonds between the biomolecule and the immobilization matrix 3 even in the case where the biomolecule to be immobilized could not be brought into proximity with the immobilization matrix 3 in a sufficiently high concentration using conventional methods.

An immobilization substrate supporting tag binding sites for use in the method of the present invention may be prepared by coupling tag binding species to activated reactive groups on the substrate. Usually, residual activated groups are then deactivated. For some tags, such as e.g. His-tags, substrate surfaces with tag binding sites, e.g. NTA, are commercially available. However, it is also possible to utilize residual activated groups which remain after coupling of the tag binding species for the covalent binding of the biomolecule to be immobilized, i.e. no further activation of reactive groups on the substrate surface is then required before binding the biomolecule to the immobilization substrate. The immobilization procedure would in this case comprise the following sequence: activate reactive groups on the immobilization substrate; couple tag binding species to the immobilization substrate via the activated reactive groups; contact biomolecules having tags with the immobilization substrate to bind the biomolecules thereto through the tags and at the same time form covalent bonds between the biomolecules and activated reactive groups.

Sensor chips prepared through the application of the method for the immobilization of biomolecules, especially proteins, of the present invention can be used as a system for detecting analytes which have affinity for the immobilized biomolecule. For example, an analytical equipment using the SPR principle, such as the above-mentioned Biacore® 3000 (Biacore AB, Uppsala, Sweden), as shown in FIG. 2, comprises a prism 4 affixed to an opposite surface of the principle surface of the base material 1, the immobilization matrix 3 being affixed to the principal surface of the base material 1, a light source 6 from which polarized light 5 is projected onto the sensor chip through the prism 4, a primary detecting element 8 onto which reflected light 7 is reflected by the metal coating 2 which reflects the polarized light 5 irradiated through the prism 4, and a flow cell 9 which is in contact with the immobilization substrate 3 upon which the protein is immobilized.

According to the principle of SPR, when polarized light 5 from the light source 6 is totally reflected by the metal coating 2, a section where the reflected light intensity is reduced can be observed on a part of the reflected light 7. The angle at which this dark section of the light appears depends on the mass (or the index of refraction) in the vicinity of the metal coating on the sensor chip. When an analyte binds to the biomolecule on the immobilization matrix 3, a mass change (=a mass increase, corresponding to an increase in refractive index) occurs, and the dark section of the light shifts from I to II. (FIG. 2). When 1 ng per 1 mm² of substance binds, it is known that the shift from I to II is 0.1 degrees. In contrast, when the mass decreases due to dissociation from the immobilization matrix, the size of the shift in the opposite direction is the same as the shift from II to I.

Therefore, by the use of the analytical equipment shown in FIG. 2, introducing the solution containing the sample into the flow cell 9, the amount of shift from I to II of the dark section of the reflected light 7 is detected by the primary detecting element 8. In this analytical equipment, the detection results may be given by taking the mass change at the surface of the sensor chip as the vertical axis and displaying the change in the measured data of mass against time (sensorgram). The units of the vertical axis may be shown as Resonance Units (RU), where 1 RU is equal to 1 pg/mm². This ratio of the change in the index of refraction is effectively the same for all biomolecules (proteins, nucleic acid, lipids), and interactions can be seen in real time without labeling the biomolecules.

By using analytical equipment utilizing this SPR principle, especially analysis of the interaction between proteins and low molecular weight compounds can be carried out, including efficient analysis of interactions between novel drug discovery targets and candidate compounds for novel drugs. Especially, sensor chips prepared through the application of the immobilization method of the present invention, being able to immobilize any kind of proteins without limitation to the category of protein, as well as making it possible to keep the proteins firmly immobilized for a considerable length of time, make it possible to carry out screening of novel drug discovery targets and candidate compounds for novel drugs using a large variety of proteins.

The present invention will be explained in further detail below by way of practical examples, but this does not limit in any way the use of this invention to these examples.

### EXAMPLES

### Comparative Example 1

In comparative example 1, a method for immobilizing a protein onto a sensor chip by a covalent bond (amine coupling) is described.

In the amine coupling method, it is necessary to find buffers whose pH values are appropriate for the preconcentration of each category of protein. This can be determined by preparing multiple solutions of protein diluted to approximately 20 μg/mL with pH 5.5, pH 5.0, pH 4.5, and pH 4.0 sodium acetate buffer of approximately 10 mM, followed by reacting each solution with the sensor chip to incite electrostatic adsorption of the protein onto the sensor chip, and then measuring the electrostatic adsorption.

In this example, a CM5 sensor chip (Biacore AB, Uppsala, Sweden) on which a carboxyl group has been introduced onto the immobilization substrate was used as the sensor chip, human serum albumin (HSA) was used as the protein, and a Biacore® 3000 (Biacore AB, Uppsala, Sweden) using the SPR principle was used as the analytical equipment.

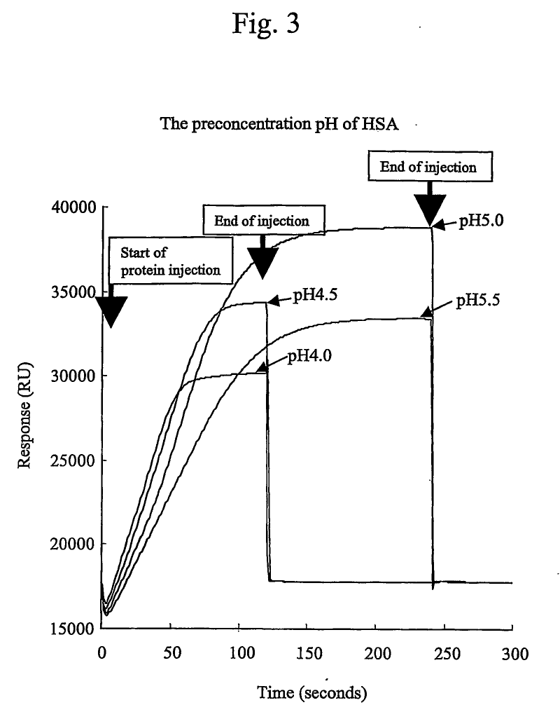

For manipulation, firstly the CM5 sensor chip was set up on the Biacore® 3000 and the system was filled with running buffer (HBS-EP or the like). Then the protein solutions diluted with each of the above-mentioned pH values of sodium acetate buffer were injected at a flow rate of about 10 μL/min for 1 to 5 minutes so that adsorption would reach steady state. The response (RU) was measured during this manipulation. The result of the measurement of RU displayed as a sensorgram is shown in FIG. 3.

Then from among the protein solutions diluted with each of the pH values of sodium acetate buffer, the one that showed an increase in RU value was chosen as the buffer appropriate for the preconcentration. Concretely, as shown in FIG. 3, from the speed of preconcentration, and from the large amount bound at the steady state, the pH 5.0 10 mM sodium acetate buffer was judged to be the appropriate buffer for the preconcentration.

From the above considerations, in comparative example 1, HSA immobilization was carried out as below by the amine coupling method by diluting HSA with pH 5.0 10 mM sodium acetate buffer. Firstly, the CM5 sensor chip was set up on the Biacore® 3000 and the system was filled with running buffer (HBS-EP or the like). Next, the system was treated for 8 minutes with a mixed solution of 0.2 M N-ethyl-N′-(dimethylaminopropyl) carbodiimide (EDC) and 0.05 M N-hydroxysuccinimide (NHS) at a flow rate of 20 μL/min. In this way, the carboxyl group on the CM5 sensor chip was activated (an active intermediate was formed). Next, HSA diluted with 10 mM sodium acetate buffer (pH 5.0) was added to the system for 7 minutes. In this way, a covalent bond was formed between the active intermediate and the amino group on the HSA, and the HSA was immobilized onto the CM5 sensor chip.

Next, the system was treated with 1 M ethanolamine for 7 minutes at a flow rate of 10 μL/min. In this way, the ethanolamine was reacted with the remaining active intermediate that had not reacted with HSA. Next, the system was washed with approximately 50 mM of sodium hydroxide for one minute at a flow rate of 20 μL/min to remove traces of HSA which had not formed a covalent bond and which remained on the CM5 sensor chip.

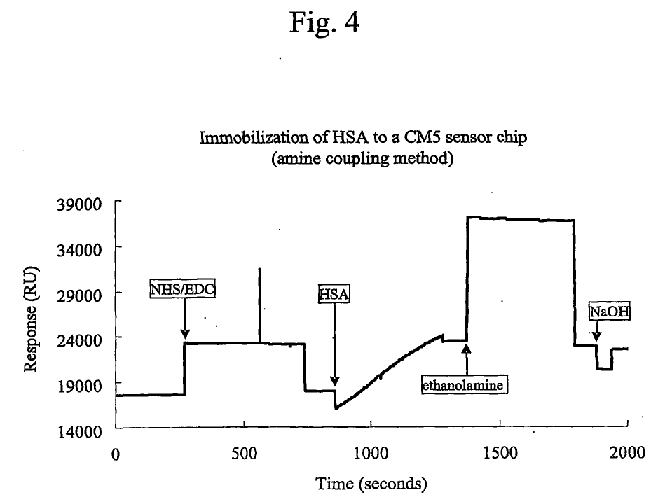

By means of the above manipulation, the amount of immobilized HSA is calculated by subtracting the response at the beginning of the immobilization from the response at the end of the immobilization, and 4944.9 RU was consistently immobilized. The sensorgram of the above operation is shown in FIG. 4.

### Comparative Example 2

Comparative example 2 was carried out in the same way as comparative example 1 except that an acidic protein was used as the protein to be immobilized. Concretely, human trypsin was used as the acidic protein.

In this example, it is necessary to find a buffer of appropriate pH for the preconcentration of human trypsin. In order to verify this in the same way as in comparative example 1, multiple solutions of human trypsin diluted to approximately 20 μg/mL with pH 5.5, pH 5.0, pH 4.5, and pH 4.0 sodium acetate buffer of approximately 10 mM were prepared.

In the same way as in comparative example 1, the response (RU) was measured using the multiple solutions prepared in this way. However, even the solution diluted with the pH 4.0 sodium acetate buffer failed to preconcentrate. Further, even if by using a solution diluted with sodium acetate buffer below pH 4.0 preconcentration is just about achieved, the pH of the above-mentioned solution is too far removed from the optimal pH (approximately pH 8) condition for the amine coupling reaction after this to occur. In this case acidic proteins will not be immobilized.

Concretely, in the case of this example, even with a solution diluted with pH 4.0 sodium acetate buffer of approximately 10 mM, the preconcentration is approximately 20 RU in 30 seconds, and immobilization is completely impossible.

### Comparative Example 3

In comparative example 3, a method for immobilizing a protein onto a sensor chip through a protein tag (His-tag) is described.

In this example, COX-2 to which a tag (His-tag) has been added to an N-terminus was used as the protein, an NTA sensor chip (Biacore AB, Uppsala, Sweden) in which nitrilotriacetic acid has been introduced onto the immobilization matrix was used as the sensor chip, and a Biacore® 3000 (Biacore AB, Uppsala, Sweden) was used as the analytical equipment.

For manipulation, firstly the NTA sensor chip was set up on the Biacore® 3000 and the system was filled with running buffer (0.005% surfactant P20, PBS or the like). Next, 0.5 M NiCl₂ was injected into the system at a flow rate of 20 μL/min for 1 minute. In this way, Ni²⁺ was trapped in the NTA on the NTA sensor chip. Next, a solution of COX-2 with the N-terminal His-tag was injected at a flow rate of 10 μL/min for 20 minutes. In this way, it was possible to immobilize the COX-2 onto the NTA sensor chip through the His-tag. In other words, COX-2 with the N-terminal His-tag was immobilized onto the NTA sensor chip due to the formation of a stable complex with the NTA bound with the Ni²⁺.

In addition, the solution containing the N-terminal His-tagged COX-2 was prepared by diluting to approximately 100 nM with the above-mentioned running buffer.

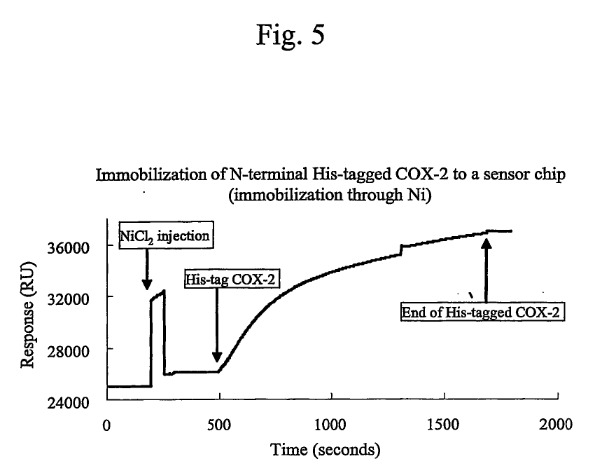

The sensorgram of the above manipulation is shown in FIG. 5. As can be seen from FIG. 5, at first 10,866 RU of the N-terminal His-tagged COX-2 was immobilized. However, immobilization of the N-terminal His-tagged COX-2 was unstable, and following that, simply by continuing the flow of running buffer, the immobilized N-terminal His-tagged COX-2 gradually separated from the chip.

### Comparative Example 4

In comparative example 4, except for the use of FK506 binding protein to which a His-tag has been added to an N-terminus (N-terminal His-tagged FKBP), the example is the same as that carried out in comparative example 3. In addition, the solution containing the N-terminal His-tagged FKBP was prepared by diluting 100 times in running buffer a lysate of bacteria in which E. coli, in which N-terminal His-tagged FKBP was expressed, had been disrupted by sonication.

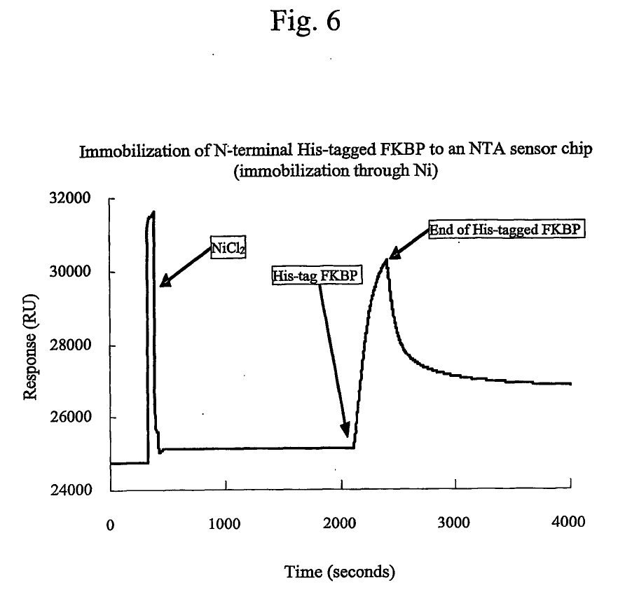

For manipulation, the immobilization was carried out in the same way as in comparative example 3 except that when immobilizing the N-terminal His-tagged FKBP, the solution containing the N-terminal His-tagged FKBP was injected at a flow rate of 10 μL/min for 5 minutes. The sensorgram of the above operation is shown in FIG. 6.

As can be seen from FIG. 6, at first 5,200 RU of the N-terminal His-tagged FKBP was immobilized, but the immobilization was unstable, and following that, simply by continuing the flow of running buffer, the immobilized N-terminal His-tagged FKBP rapidly separated from the NTA sensor chip such that 20 minutes later the value had declined to 1766.2 RU.

### Comparative Example 5

In comparative example 5, an analysis was carried out of the interaction between a compound and N-terminal His-tagged COX-2 NTA sensor chip, as prepared in comparative example 3, when N-terminal His-tagged COX-2 was reacted with a low molecular weight compound. NS398, known to be a selective inhibitor of COX-2, was used as the low molecular weight compound.

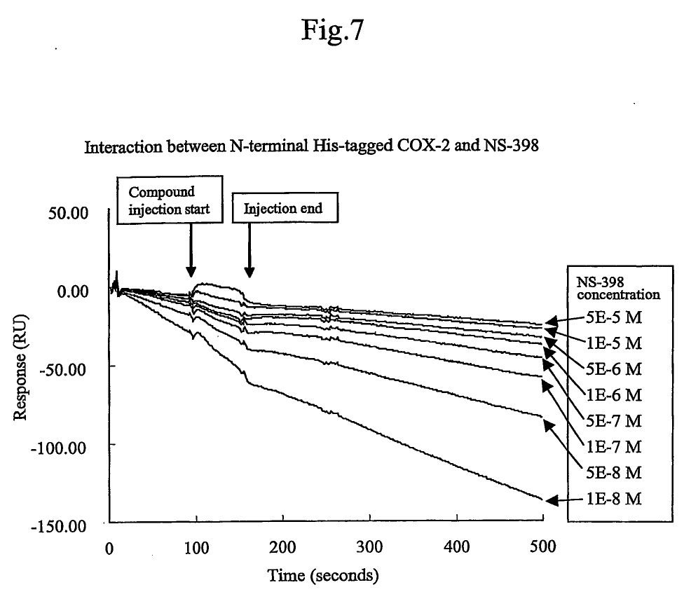

For manipulation, firstly the NTA sensor chip as in comparative example 3 was set up on the Biacore® 3000 and the system was filled with running buffer (5% DMSO, 0.005% surfactant P20, PBS or the like). In this state, NS398 in gradually rising concentrations from 1×10⁻⁸ M was injected repeatedly (flow rate 10 μL/min for 1 minute). The resulting sensorgrams for the injection of each concentration of NS398 are shown superimposed in FIG. 7. In FIG. 7, the response for each concentration at the start of the injection (0 time) was superimposed on 0.

Injecting NS398 at low concentrations and moving up in order to high concentrations of NS398, above a concentration of 1×10⁻⁵ M bonding was seen between the N-terminal His-tagged COX-2 immobilized on the NTA sensor chip and NS398. This bonding was observed under the presence of NS398 and rapidly dissociated at the completion of injection. However, as shown in FIG. 7, as the baseline fell, the results of the injection of each concentration could not be superimposed, and it was very difficult to analyze the affinity.

### Comparative Example 6

In comparative example 6, an analysis was carried out of the interaction between a compound and N-terminal His-tagged FKBP NTA sensor chip, as prepared in comparative example 4, when N-terminal His-tagged FKBP was reacted with a low molecular weight compound. FK506, known to bind with FKBP, was used as the low molecular weight compound.

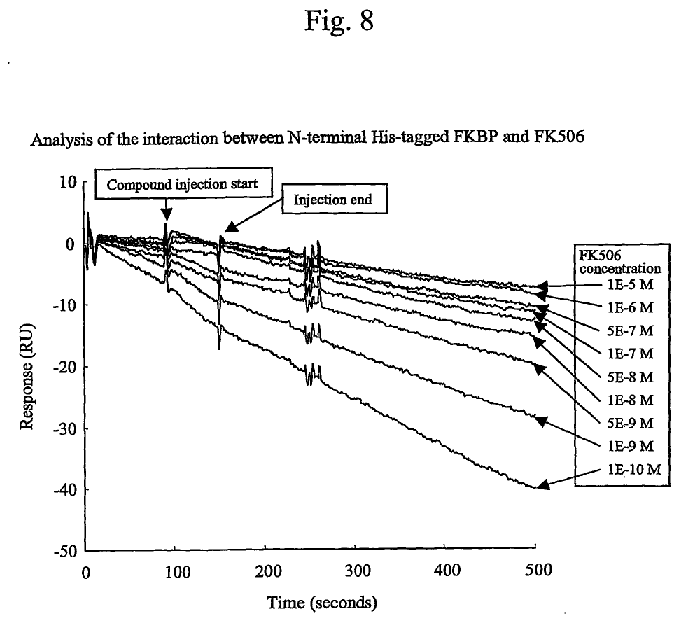

For manipulation, firstly the NTA sensor chip as in comparative example 4 was set up on the Biacore® 3000 and the system was filled with running buffer (5% DMSO, 0.005% surfactant P20, PBS or the like). In this state, FK506 in gradually rising concentrations from 1×10⁻⁸ M was injected repeatedly (flow rate 10 μL/min for 1 minute). The resulting sensorgrams for the injection of each concentration of FK506 are shown superimposed in FIG. 8. In FIG. 8, the response for each concentration at the start of the injection (0 time) was superimposed on 0.

However, binding between the N-terminal His-tagged FKBP immobilized on the NTA sensor chip and FK506 was almost completely absent, as seen from the results shown in FIG. 8.

### Practical Example 1

In practical example 1, a method for immobilizing protein by a covalent bond (amine coupling) and a tag to a sensor chip by application of this invention is described. In this example, FKBP to which a His-tag has been added to an N-terminus was used as the protein, an NTA sensor chip (Biacore AB, Uppsala, Sweden) in which nitrilotriacetic acid has been introduced onto the immobilization matrix was used as the sensor chip, and a Biacore® 3000 (Biacore AB, Uppsala, Sweden) was used as the analytical equipment.

Firstly, the NTA sensor chip was set up on the Biacore® 3000 and the system was filled with running buffer (0.005% surfactant P20, PBS pH 7.4 or the like). Next, the system was treated for 7 minutes with a mixed solution of 0.2 M N-ethyl-N′-(dimethylamino-propyl) carbodiimide (EDC) and 0.05 M N-hydroxysuccinimide (NHS) at a flow rate of 10 μL/min. In this way, the carboxyl group on the NTA sensor chip was activated (an active intermediate was formed). At this time, it is thought that the carboxyl group on the carboxymethyldextran, which is the immobilization matrix on the NTA sensor chip, and a portion of the carboxyl groups on the NTA become active intermediates, but it is thought that the remaining unreacted portion of the NTA is sufficient to form a complex afterwards between the Ni²⁺ and the N-terminal His-tagged FKBP.

Next, 0.5 M NiCl₂ was injected into the system at a flow rate of 20 μL/min for 1 minute. In this way, Ni²⁺ was trapped in the NTA on the NTA sensor chip. Next, a solution containing N-terminal His-tagged FKBP was injected into the system at a flow rate of 10 μL/min for 20 minutes. In this way, the N-terminal His-tagged FKBP, by forming a complex with the NTA bound to the Ni²⁺, was concentrated on the NTA sensor chip, covalent bonds being formed efficiently with the active intermediate, and was thereby fly immobilized onto the NTA sensor chip. In addition, the solution containing the N-terminal His-tagged FKBP was prepared by diluting 100 times in running buffer a lysate of bacteria in which E. coli expressing N-terminal His-tagged FKBP had been disrupted by sonication.

Next, 1 M ethanolamine was injected into the system for 7 minutes at a flow rate of 10 μL/min. In this way, remaining unreacted active intermediate was reacted with ethanolamine to terminate the immobilization reaction. Next, approximately 50 mM sodium hydroxide was injected into the system for one minute at a flow rate of 20 μL/min. In this way, the NTA sensor chip was washed, and further, traces of N-terminal His-tagged FKBP which had not formed a covalent bond and which remained on the NTA sensor chip were removed.

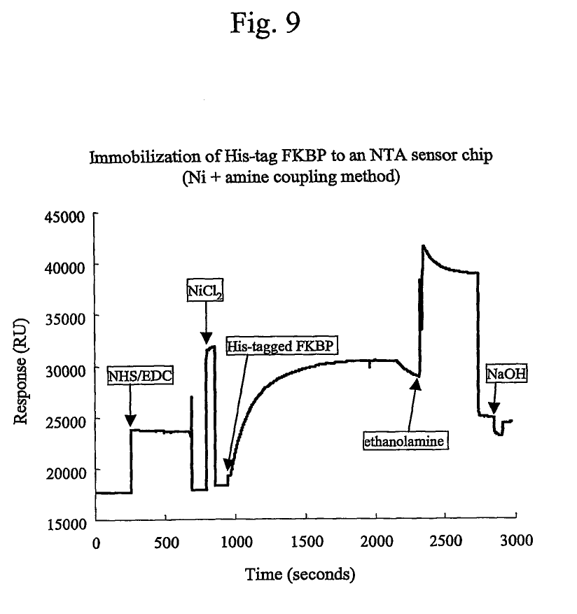

The sensorgram of the above manipulation is shown in FIG. 9. At first 12,664 RU of the N-terminal His-tagged FKBP had bound to the NTA on the NTA sensor chip through affinity with Ni²⁺. Following this, even after treatment with ethanolamine and washing treatment with sodium hydroxide, N-terminal His-tagged FKBP did not dissociate and 6732.2 RU were immobilized on the sensor chip. This is because amine coupling (covalent bond) was formed almost simultaneously to the N-terminal His-tagged FKBP binding to the NTA through affinity with the Ni²⁺.

Comparing practical example 1 with comparative example 4, it is clear that after activating the carboxyl group on the NTA sensor chip, as well as binding the FKBP to the NTA sensor chip through the His-tag, because the FKBP was immobilized on the NTA sensor chip through a covalent bond it was possible to immobilize the FKBP more firmly.

### Practical Example 2

In practical example 2, a method for immobilizing protein similar to that in practical example 1, except that N-terminal His-tagged COX-2 was used as the protein, is described. In addition, the solution containing the N-terminal His-tagged COX-2 was prepared by diluting to approximately 100 nM with the above-mentioned running buffer.

In this example, when immobilizing the N-terminal His-tagged COX-2, the solution containing the N-terminal His-tagged COX-2 was injected at a flow rate of 10 μL/min for approximately 30 minutes. Following this, in the same way as in practical example 1, the immobilization reaction was terminated by reaction with ethanolamine and then washed using approximately 50 mM sodium hydroxide.

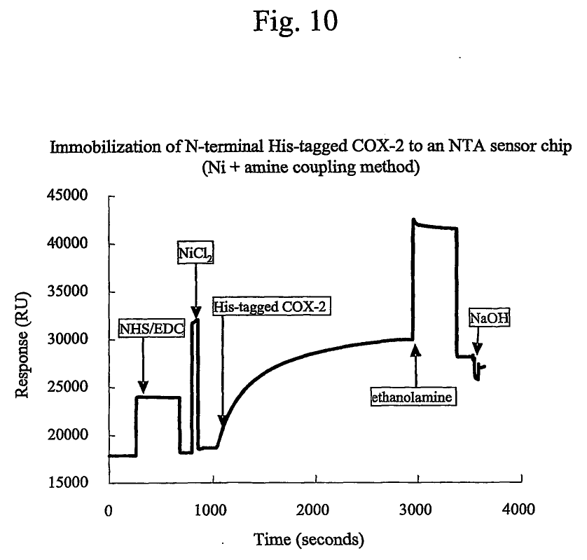

The sensorgram of the above manipulation is shown in FIG. 10. As can be seen in FIG. 10, there was a stable immobilization of 9,219 RU of N-terminal His-tagged COX-2. Further, in this example as well, even after treatment with ethanolamine and washing treatment with sodium hydroxide, N-terminal His-tagged COX-2 did not dissociate and was firmly immobilized on the NTA sensor chip.

### Practical Example 3

In practical example 3, a method for immobilizing a protein to a sensor chip through a covalent bond (amine coupling) and a tag by application of this invention is described. In this example, Cyclophilin A to which an His-tag has been added to an N-terminus was used as the protein, a CM5 sensor chip (Biacore AB, Uppsala, Sweden) to which an anti-His tag antibody has been immobilized was used as the sensor chip, and a Biacore® 3000 (Biacore AB, Uppsala, Sweden) was used as the analytical equipment.

Firstly, the CM5 sensor chip to which an anti-His tag antibody had been immobilized (hereafter anti-His tag antibody sensor chip) was set up on the Biacore® 3000 and the system filled with running buffer (HBS-EP; Biacore AB, Uppsala, Sweden). Further, immobilization of the anti-His tag antibody to the sensor chip was easily carried out by the amine coupling method, and in this practical example approximately 10,000 RU of anti-5×His tag antibody (Qiagen, Valencia, Calif., U.S.A.) were immobilized.

Next, the carboxyl group on the anti-His tag antibody sensor chip was activated (an active intermediate was formed) by treatment for 4 minutes with a mixed solution of 0.2 M N-ethyl-N′-(dimethylaminopropyl) carbodiimide (EDC) and 0.05 M N-hydroxysuccinimide (NHS) at a flow rate of 10 μL/min. At this time, it is thought that the carboxyl group on the carboxymethyldextran, and a portion of the carboxyl groups on the antibody become active intermediates, but it is thought that the remaining unreacted portion of the antibody is sufficient to form a bond between the remaining antibody and the His-tagged protein.

Next, a diluted solution of N-terminal His-tagged Cyclophilin A was injected into the system at a flow rate of 10 μL/min for approximately 30 minutes. In this way, the protein with the His tag was firmly immobilized onto the sensor chip by being concentrated on the sensor chip by the formation of affinity bonds with the anti-His-tag antibody, covalent bonds then being formed efficiently with the active intermediate. Further, the solution containing the N-terminal His-tagged Cyclophilin A was prepared by diluting with running buffer a lysate of bacteria in which E. coli expressing N-terminal His-tagged Cyclophilin A had been disrupted by sonication.

Next, 1 M ethanolamine was injected into the system for 7 minutes at a flow rate of 10 μL/min in order to react the remaining unreacted active intermediate with the ethanolamine to terminate the immobilization reaction. Next, the system was treated with pH 1.5 glycine-hydrochloric acid buffer solution for one minute. In this way, the CM5 sensor chip was washed, and further, traces of N-terminal His-tagged Cyclophilin A or the like which had not formed a covalent bond and which remained on the CM5 sensor chip were removed.

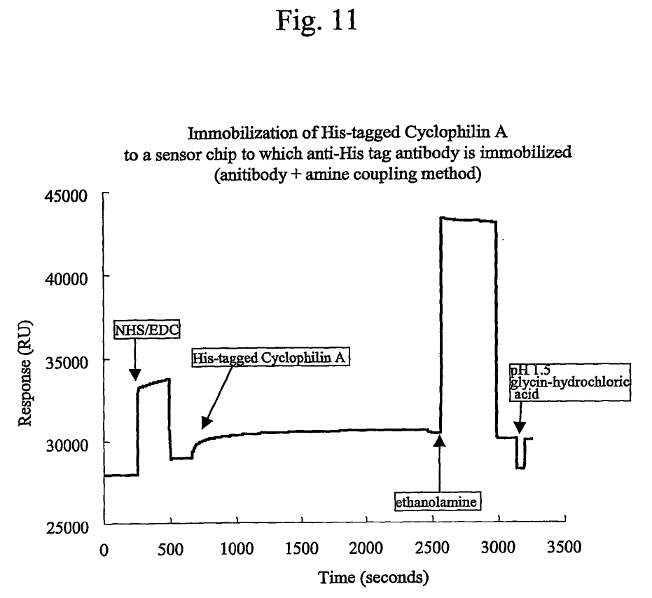

The sensorgram of the above operation is shown in FIG. 11. From FIG. 11 it is seen that 1,644 RU of the N-terminal His-tagged Cyclophilin A had at first bound to the antibody through affinity. Following this, even after treatment with ethanolamine and washing treatment with glycine-hydrochloric acid buffer solution, N-terminal His-tagged Cyclophilin A did not dissociate and 1,049 RU were immobilized on the sensor chip. This is because amine coupling (covalent bond) was formed almost simultaneously to the N-terminal His-tagged Cyclophilin A binding to the anti-His antibody.

### Practical Example 4

In practical example 4, a method for immobilizing protein similar to that in practical example 1, except that N-terminal His-tagged Akt1/PKBa (Upstate Biotechnology, Waltham, Mass., U.S.A.; product name 14-341) was used as the protein, is described. Akt1/PKBa is known to be a serine/threonine protein kinase. Akt1/PKBa was used after removing imidazole by applying the commercial solution to a desalting column.

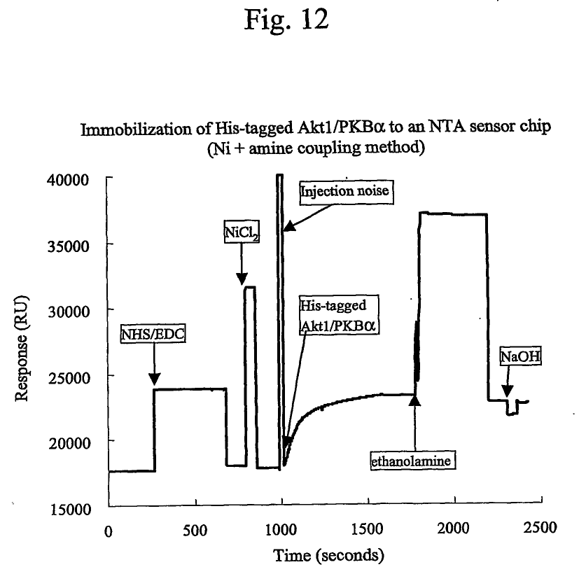

In this example, as in practical example 1, N-terminal His-tagged Akt1/PKBa was immobilized, the immobilization reaction was terminated by reaction with ethanolamine, and then washed using approximately 50 mM sodium hydroxide. The sensorgram of the above operation is shown in FIG. 12. As seen in FIG. 12, there was stable immobilization of 5018.7 RU of N-terminal His-tagged Akt1/PKBa. Further, in this example as well, even after treatment with ethanolamine and washing treatment with sodium hydroxide, N-terminal His-tagged Akt1/PKBa did not dissociate and was firmly immobilized on the NTA sensor chip.

### Practical Example 5

In practical example 5, a method for immobilizing protein similar to that in practical example 1, except that N-terminal His-tagged MSK1 (Upstate Biotechnology, Waltham, Mass., U.S.A.; product name 14-438) was used as the protein, is described. MSK1 is known to be a serine/threonine protein kinase.

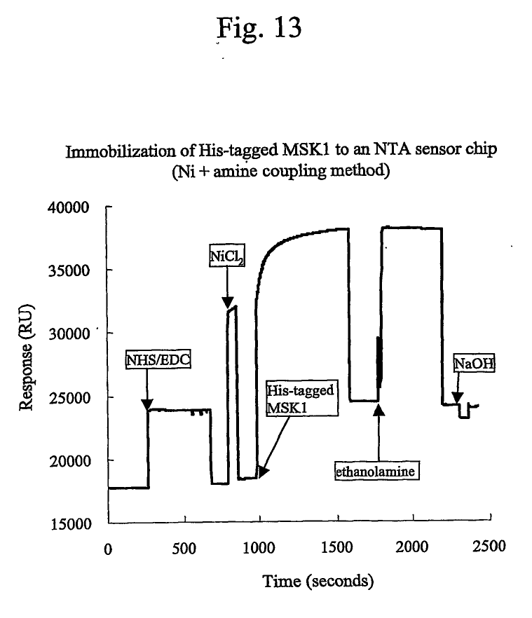

In this example, as in practical example 1, N-terminal His-tagged MSK1 was immobilized, the immobilization reaction was terminated by reaction with ethanolamine, and then washed using approximately 50 mM sodium hydroxide. The sensorgram of the above manipulation is shown in FIG. 13. As seen in FIG. 13, there was stable immobilization of 6232.3 RU of N-terminal His-tagged MSK1. Further, in this example as well, even after treatment with ethanolamine and washing treatment with sodium hydroxide, N-terminal His-tagged MSK1 did not dissociate and was firmly immobilized on the NTA sensor chip.

### Practical Example 6

In practical example 6, a method for immobilizing protein similar to that in practical example 1, except that N-terminal His-tagged PKA (Upstate Biotechnology, Waltham, Mass., U.S.A.; product name 14-440) was used as the protein, is described. PKA is known to be a serine/threonine protein kinase.

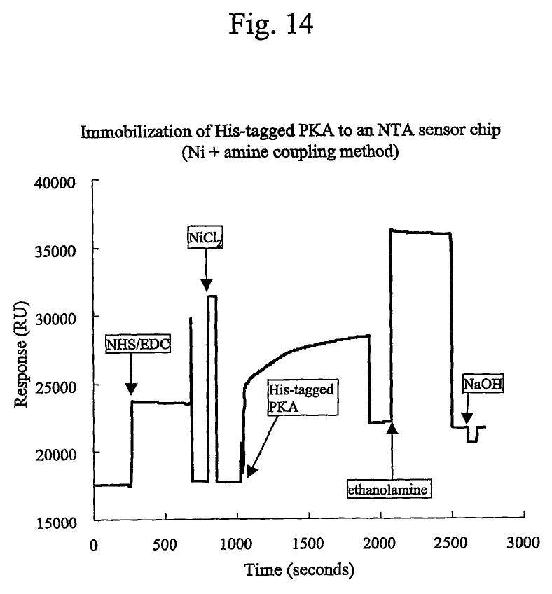

In this example, as in practical example 1, N-terminal His-tagged PKA was immobilized, the immobilization reaction was terminated by reaction with ethanolamine, and then washed using approximately 50 mM sodium hydroxide. The sensorgram of the above operation is shown in FIG. 14. As seen in FIG. 14, there was stable immobilization of 4,134.5 RU of N-terminal His-tagged PKA. Further, in this example as well, even after treatment with ethanolamine and washing treatment with sodium hydroxide, N-terminal His-tagged PKA did not dissociate and was firmly immobilized on the NTA sensor chip.

### Practical Example 7

In practical example 7, a method for immobilizing protein similar to that in practical example 1, except that N-terminal His-tagged PRAK (Upstate Biotechnology, Waltham, Mass., U.S.A.; product name 14-334) was used as the protein, is described. PRAK is known to be a serine/threonine protein kinase.

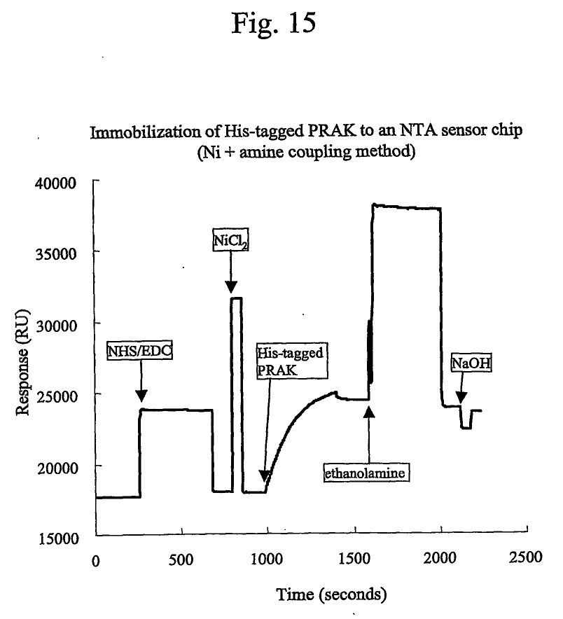

In this example, as in practical example 1, N-terminal His-tagged PRAK was immobilized, the immobilization reaction was terminated by reaction with ethanolamine, and then washed using approximately 50 mM sodium hydroxide. The sensorgram of the above operation is shown in FIG. 15. As seen in FIG. 15, there was a stable immobilization of 5,869.6 RU of N-terminal His-tagged PRAK. Further, in this example as well, even after treatment with ethanolamine and washing treatment with sodium hydroxide, N-terminal His-tagged PRAK did not dissociate and was firmly immobilized on the NTA sensor chip.

### Practical Example 8

In practical example 8, a method for immobilizing protein similar to that in practical example 1, except that N-terminal His-tagged ROKα/ROCK-II (Upstate Biotechnology, Waltham, Mass., U.S.A.; product name 14-338) was used as the protein, is described. ROKα/ROCK-II is known to be a serine/threonine protein kinase.

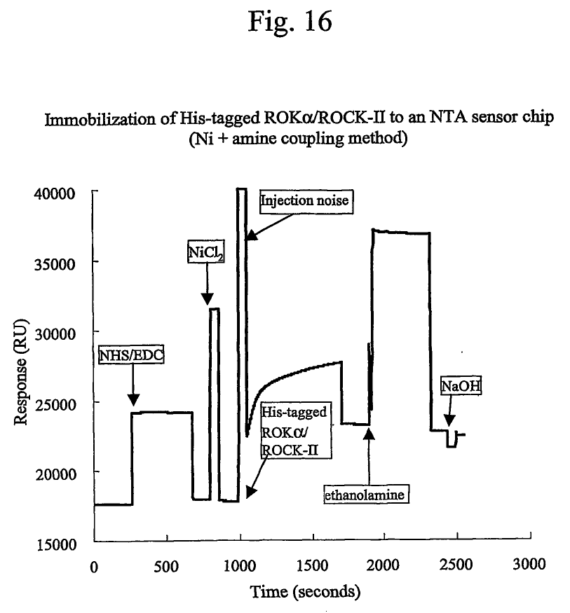

In this example, as in practical example 1, N-terminal His-tagged ROKα/ROCK-II was immobilized, the immobilization reaction was terminated by reaction with ethanolamine, and then washed using approximately 50 mM sodium hydroxide. The sensorgram of the above operation is shown in FIG. 16. As seen in FIG. 16, there was stable immobilization of 4,775.5 RU of N-terminal His-tagged ROKα/ROCK-II. Further, in this example as well, even after treatment with ethanolamine and washing treatment with sodium hydroxide, N-terminal His-tagged ROKα/ROCK-II did not dissociate and was firmly immobilized on the NTA sensor chip.

### Practical Example 9

In practical example 9, an analysis was carried out of the interaction between a low molecular weight compound and N-terminal His-tagged FKBP when the compound is reacted with an NTA sensor chip, as prepared in practical example 1. FK506, known to bind with FKBP, was used as the low molecular weight compound.

For manipulation, firstly the NTA sensor chip as described in practical example 1 was set up on the Biacore® 3000 and the system was filled with running buffer (0.005% P20, 5% DMSO, PBS pH 7.4). In this state, FK506 in gradually rising concentrations from 5×10⁻¹⁰ M was injected repeatedly (flow rate 50 μL/min for 1 minute). In addition, after the injection of each concentration, 10 mM glycine-HCl pH 1.5 was injected for 30 seconds in order to dissociate FK06 and regenerate FKBP.

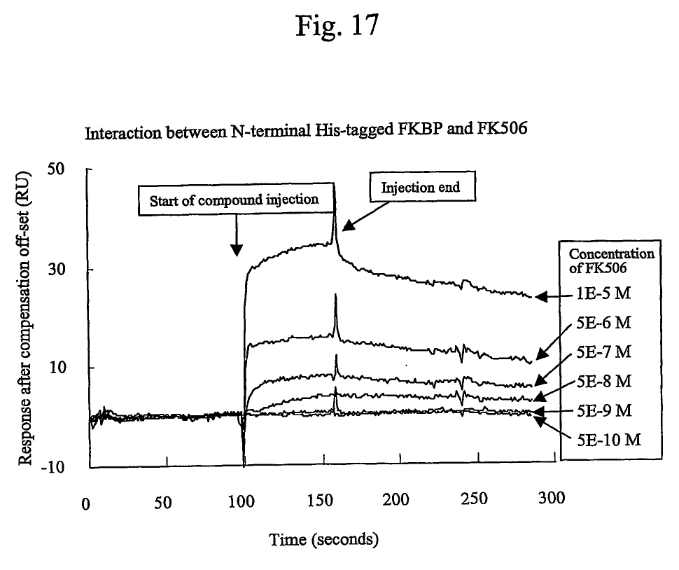

The resulting sensorgrams for the injection of each concentration of FK506 are shown superimposed in FIG. 17. In FIG. 17, the response for each concentration at the start of the injection (0 time) was superimposed on 0.

Injecting FK506 at low concentrations and moving up in order to high concentrations of FK506, above a concentration of 5×10⁻⁸ M binding was seen between the N-terminal His-tagged FKBP immobilized on the NTA sensor chip and FK506. Further, choosing 1×10⁻⁵ M and 5×10⁻⁶ M, whose responses were above 10 RU, when the binding constant was calculated, the binding constant for N-terminal His-tagged FKBP and FK506 was 3×10⁻⁹ M, slightly higher than the literature value (0.4 nM). Taking into account the effect of measurement temperature or the like it was thought that a specific binding had been detected.

### Practical Example 10

In practical example 10, an analysis was carried out of the interaction between a compound and N-terminal His-tagged COX-2 when a low molecular weight compound was reacted with an NTA sensor chip, as prepared in practical example 2. NS-398, known to bind with COX-2, was used as the low molecular weight compound.

For manipulation, firstly the NTA sensor chip as in practical example 2 was set up on the Biacore® 3000 and the system was filled with running buffer (0.005% P20, 5% DMSO, PBS pH 7.4). In this state, NS-398 in gradually rising concentrations from 5×10⁻⁸ M was injected repeatedly (flow rate 50 μL/min for 1 minute). In addition, after the injection of each concentration, 10 mM glycine-HCl pH 2.0 was injected at a flow rate of 50 μL/min for 30 seconds in order to dissociate NS-398 and regenerate COX-2. In addition, even without carrying out the regeneration manipulation, the NS-398 and COX-2 binding rapidly dissociates following the end of injection of NS-938 and the regeneration operation conditions were therefore made to be comparatively mild.

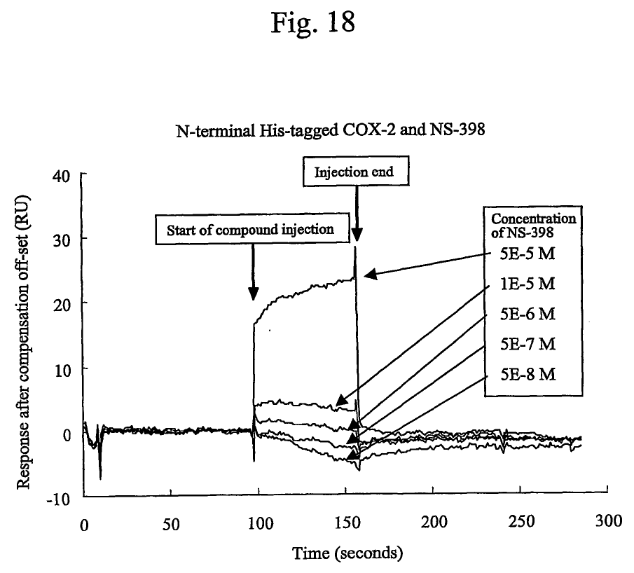

The resulting sensorgrams for the injection of each concentration of NS-398 are shown superimposed in FIG. 18. In FIG. 18, the response for each concentration at the start of the injection (0 time) was superimposed on 0.

Injecting NS-398 at low concentrations and moving up in order to high concentrations of NS-398, above a concentration of 5×10⁻⁶ M binding was seen between the N-terminal His-tagged COX-2 immobilized on the NTA sensor chip and NS-398. Further, choosing 5×10⁻⁵ M, 1×10⁻⁶ M, and 5×10⁻⁶ M, when the binding constant was calculated, the binding constant for N-terminal His-tagged COX-2 and NS-398 was K_{d}=5×10⁻⁴ M. The literature gives the value for Kᵢ for NS-398 and COX-2 as 11.50 μM, and it was thought that the result of this analysis corresponded thereto.

### Practical Example 11

In practical example 11, the interaction between N-terminal His-tagged Cyclophilin A and a low molecular weight compound was analyzed by reacting the low molecular weight compound with the CM5 sensor chip prepared in practical example 3. Cyclosporine A, which is known to bind to Cyclophilin A, was used as the low molecular weight compound.

In this manipulation, firstly the CM5 sensor chip as in practical example 3 was set up on the Biacore® 3000, the system of which was filled with running buffer (5% DMSO, HBS-EP buffer solution). In this state Cyclosporine A was repeatedly injected (flow rate 50 μL/min for one minute) starting at a concentration of 5×10⁻⁸ M and gradually working up to higher concentrations. Further, regeneration operations were not carried out as the binding between Cyclophilin A and Cyclosporine A rapidly dissociates following the end of injection without carrying out a regeneration operation.

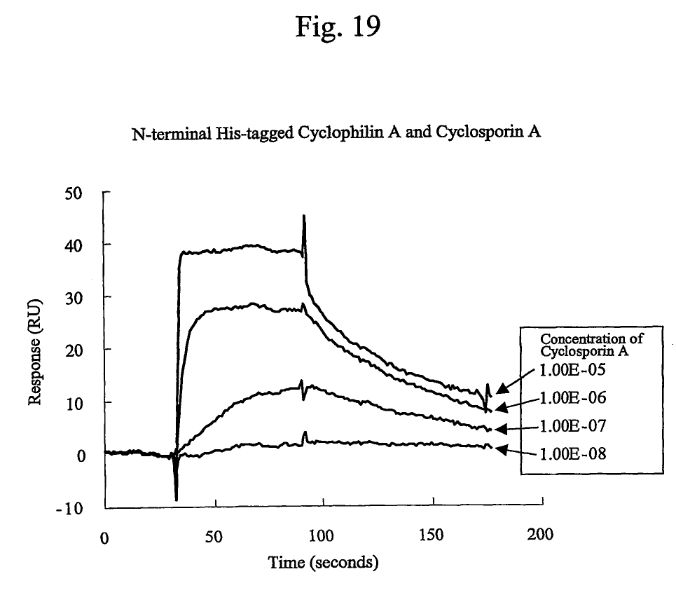

Sensorgrams for the injections of each of the different concentrations of Cyclosporine A are shown superimposed on one another in FIG. 19. In FIG. 19, the response for each concentration at the start of the injection (zero time) was superimposed on zero.

Injecting Cyclosporine A at low concentrations and moving up in order to high concentrations of Cyclosporine A, above a concentration of 1×10⁻⁸ M binding was seen between the N-terminal His-tagged Cyclophilin A immobilized on the CM5 sensor chip and Cyclosporine A. Further, when the binding constant was calculated choosing the results up to 1×10⁻⁵ M, the binding constant for N-terminal His-tagged Cyclophilin A and Cyclosporine A was K_{d}=8.8×10⁻⁸ M, which is a close match with the literature value.

### Practical Example 12

In practical example 12 a method for immobilizing a protein to a sensor chip through a covalent bond (amine coupling) and a tag by application of this invention is described. In this example, mouse IgG was used as the protein, a CM5 sensor chip (Biacore AB, Uppsala, Sweden) was used as the sensor chip and a Biacore® 3000 (Biacore AB, Uppsala, Sweden) was used as the analytical equipment.

Firstly, the CM5 sensor chip was set up on the Biacore® 3000 and the system was filled with running buffer (HBS-EP, Biacore AB, Uppsala, Sweden). Carboxylic groups of the carboxymethyldextran layer on the sensor chip surface were activated by treatment for 10 minutes with a mixed solution of 0.2 M N-ethyl-N′-(dimethylaminopropyl) carbodiimide (EDC) and 0.05 M N-hydroxysuccinimide (NHS) at a flow rate of 10 μl/min. Next, a diluted solution of Protein A (10 μg/ml in 10 mM sodium acetate buffer, pH 4.0) was injected into the system at a flow rate of 10 μl/min for 5 minutes. In this way, Protein A becomes covalently immobilized onto the sensor chip. Further, a solution of mouse IgG (10 μg/ml in running buffer) was injected at 10 μl/min for 5 minutes directly after the Protein A solution and was thereby firmly immobilized on the sensor chip surface by being concentrated on the sensor chip through the formation of affinity bonds via Protein A-IgG interaction, and covalent bonds then being formed efficiently to residual activated carboxylic groups on the carboxymethyldextran layer.

Next, 1 M ethanolamine was injected into the system for 10 minutes at a flow rate of 10 μl/min in order to react remaining unreacted active intermediate with the ethanolamine to terminate the immobilization reaction.

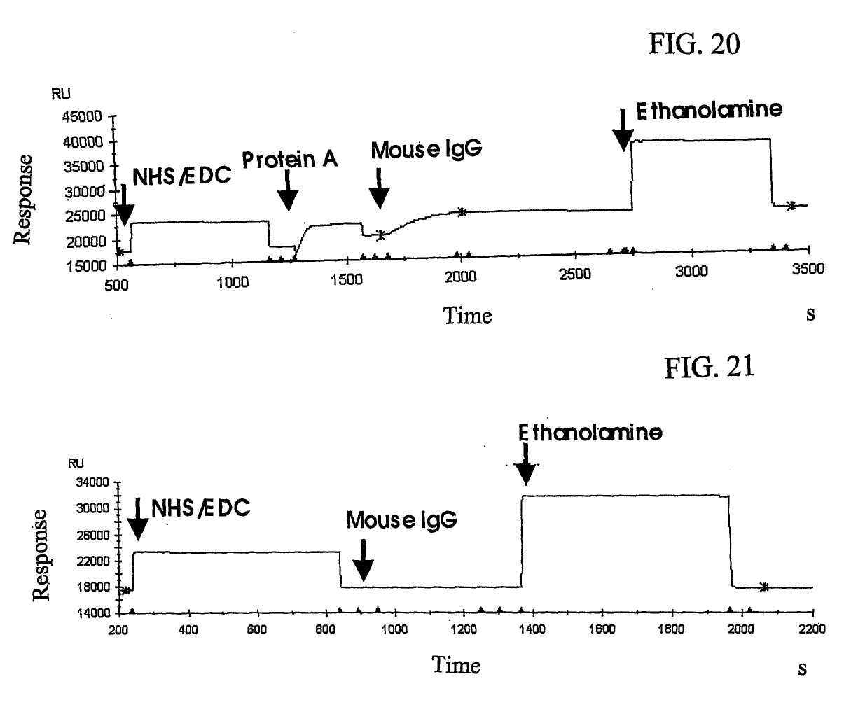

The sensorgram of the above operation is shown in FIG. 20. From FIG. 20 it is seen that 2,017 RU of Protein A had first been covalently immobilized to the sensor surface. Following this, 4,160 RU of mouse IgG had bound to the sensor surface through a combination of affinity and covalent attachment. Following this, even after treatment with ethanolamine, mouse IgG did not dissociate significantly and 3,920 RU of mouse IgG remained bound to the sensor chip.

In a comparative experiment, the injection of Protein A was excluded and a mouse IgG solution with identical composition as described above was injected directly after the EDC/NHS activation pulse. A 1 M ethanolamine pulse was thereafter injected, in the same way as described above. The sensorgram of this comparative experiment is shown in FIG. 21. From FIG. 21 it is seen that only diminutive amounts of mouse IgG were covalently immobilized, After the treatment with ethanolamine, 250 RU of mouse IgG remained bound to the sensor chip.

### Practical Example 13

In practical example 13, the interaction between a mouse IgG antibody and an anti-mouse IgG was analysed by reacting the anti-mouse IgG with the CM5 sensor chip prepared as in practical example 12.

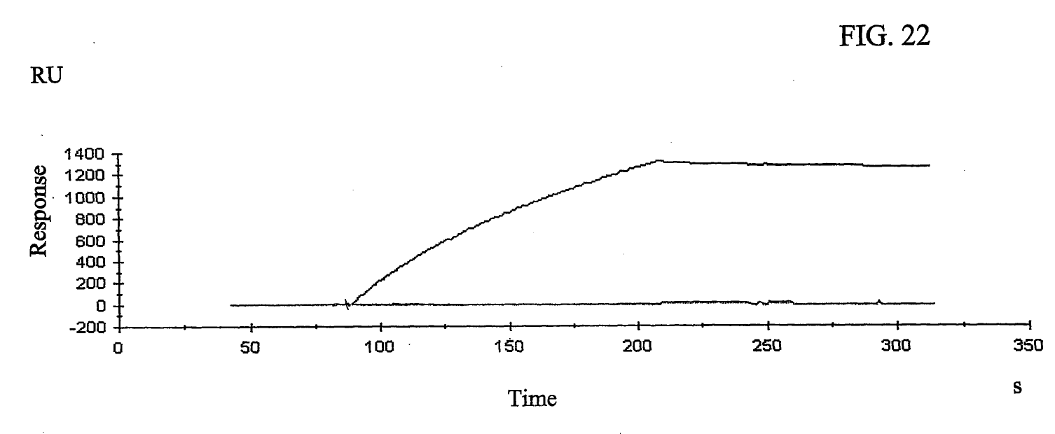

In this manipulation, firstly the CM5 sensor chip prepared as in practical example 12 was set up in a Biacore® 3000, the system was filled with running buffer (HBS-EP; Biacore AB, Uppsala, Sweden). In this state, anti-mouse IgG was injected at a flow rate of 10 μl/min for 2 minutes. A sensorgram for the injection is shown in FIG. 22. In FIG. 22, it is seen that 1,290 RU of the anti-mouse IgG bound to the sensor surface (upper curve).

Superimposed in the same FIG. 22 is shown the sensorgram of the injection of the anti-mouse IgG on the CM5 sensor chip prepared as in the comparative experiment described in practical example 12 above. In this comparative experiment on the sensor surface prepared without the capturing Protein A molecule, it is seen from FIG. 22 that only 4 RU of anti-mouse IgG was bound to the sensor surface (lower curve). This diminutive binding shows that the observed binding of anti-mouse IgG in the example with Protein A as a first capture molecule is specific.

Effects of the Invention

As explained in detail above, the method for the immobilization of biomolecules of the present invention comprises activating a reactive group which is capable of forming a covalent bond with the biomolecule to be immobilized, and after that causing a tag on the protein to be immobilized to interact with the immobilization matrix to form a covalent bond between the reactive group on the immobilization matrix and the biomolecule to be immobilized. By use of the method for the immobilization of biomolecules of the present invention, it is possible to immobilize all biomolecules that have a tag, and it is also possible to firmly immobilize the biomolecule to be immobilized on the immobilization matrix for a long length of time.

1. A method for immobilizing biomolecules, which method comprises contacting a solution containing a biomolecule or biomolecules provided with at last one tag with an immobilization substrate which has (i) binding sites for the biomolecule tag or tags, and (ii) activated reactive groups which are capable of forming covalent bonds with the biomolecule or biomolecules.

2. The method according to claim 1, comprising the steps of:
a first step wherein the reactive groups of the immobilization substrate which are capable of forming a covalent bond with the biomolecule or biomolecules to be immobilized are activated;
a second step wherein a solution containing the biomolecule or biomolecules to be immobilized is reacted with the immobilization substrate following the first step, and
wherein, in the second step, the biomolecule or biomolecules are immobilized on the immobilization substrate through interaction between the tag or tags and tag-binding sites of the immobilization substrate and covalent bonds formed between the reactive groups and the biomolecule or biomolecules.

3. The method according to claim 2, wherein the reactive groups are carboxyl groups, and in the second step, an amine coupling is formed between the carboxyl groups and an amino group on the biomolecule to be immobilized.

4. The method according to claim 2, wherein the tag is a histidine tag, and in the second step, an interaction is effected between the histidine tag and the immobilization substrate.

5. The method according to claim 4, wherein, in the second step, an interaction is effected between the histidine tag and the immobilization substrate through a complex.

6. The method according to claim 5, wherein, in the second step, an interaction is effected between the histidine tag and the immobilization substrate through a metal ion chelate.

7. The method according to claim 6, wherein, in the second step, an interaction is effected between the histidine tag and the immobilization substrate through Ni²⁺ nitrilotriacetic acid (Ni-NTA).

8. The method according to claim 6, wherein, in the second step, an interaction is effected between the histidine tag and the immobilization substrate through Ni²⁺ iminodiacetic acid (Ni-IDA).

9. The method according to claim 1, wherein the tag-binding site of the immobilization substrate is an antibody to the tag.

10. The method according to claim 9, wherein the tag is a histidine tag, the antibody is an anti-histidine antibody and, in the second step, an intereaction is effected between the histidine tag and the immobilization substrate through an anti-histidine antibody.

11. The method according to claim 1, wherein the tag is an inherent part of a native biomolecule.

12. The method according to claim 1, wherein the biomolecule is a protein.

13. A method for determining biomolecule-low molecular weight compound affinity and/or kinetics comprising:
a step for reacting a sample containing a low molecular weight compound or compounds to be determined with an immobilization substrate to which a biomolecule or biomolecules have been immobilized using the method for immobilizing biomolecules as defined in claim 1, and
a step for determining the affinity and/or kinetics of the low molecular weight compound or compounds contained in the sample for the biomolecule or biomolecules immobilized on the immobilization substrate.

14. The method according to claim 13, wherein the affinity and/or kinetics of a biomolecule and a low molecular weight compound is determined using the principle of surface plasmon resonance (SPR) in the step for determining affinity and/or kinetics.

15. The method according to claim 13, wherein the biomolecule is a protein.

16. A method for determining protein-protein affinity and/or kinetics comprising:
a step for reacting a sample containing a protein or proteins to be determined with an immoibilization substrate which has a protein or proteins immobilized thereon using the method for immobilizing biomolecules as defined in claim 1, and
a step for determining the affinity and/or kinetics of the protein or proteins contained in the sample for the protein or proteins immobilized on the immobilization substrate.

17. The method according to claim 16, wherein the affinity and/or kinetics of a protein in the sample for an immobilized protein is determined using the principle of surface plasmon resonance (SPR) in the step for determining the affinity and/or kinetics.

18. An immobilization substrate comprising at least one immobilized biomolecule, wherein the biomolecule or biomolecules have been immobilized by the method defined in claim 1.

19. The immobilization substrate of claim 18, which comprises:
a substrate, and
polysaccharide chains arranged on the substrate, into which are introduced reactive groups capable of forming covalent bonds with a biomolecule or biomolecules to be immobilized thereon,
wherein the biomolecule or biomolecules interact with the polysaccharide chain through a chelate and form covalent bonds with the reactive groups.

20. The method according to claim 18, wherein the biomolecule is a protein.
